(19) United States
(12) Patent Application Publication
(10) Pub. No.: US 2019/0029750 A1
(43) Pub. Date: Jan. 31, 2019

(54) DIRECTIONAL BALLOON TRANSSEPTAL INSERTION DEVICE FOR MEDICAL PROCEDURES

(71) Applicant: East End Medical LLC, Lewes, DE (US)

(72) Inventor: BRIJESHWAR S. MAINI, West Palm Beach, FL (US)

(21) Appl. No.: 16/047,910

(22) Filed: Jul. 27, 2018

Related U.S. Application Data
(60) Provisional application No. 62/538,552, filed on Jul. 28, 2017.
(60) Provisional application No. 62/592,061, filed on Nov. 29, 2017.

(51) Int. Cl. A61B 18/14; A61B 17/34; A61M 25/10

(52) U.S. Cl. CPC A61B 18/1492; A61B 17/3478; A61B 2017/00243; A61B 17/3494; A61M 25/10

(57) ABSTRACT

A transseptal insertion device suitable for facilitating precise and safe transseptal puncture of a cardiac interatrial septum includes a sheath that defines a lumen and has a distal end that is closest to the cardiac interatrial septum of a patient and a proximal end that is external to the patent, a balloon that is connected to the distal end of the sheath, in which the balloon, when inflated, overhangs and extends past the distal end of the sheath, preventing accidental puncturing of the cardiac interatrial septum and stabilizing the transseptal insertion device against fossa ovalis of the cardiac interatrial septum, and a dilator that is positioned within the at least one lumen. The dilator has a distal end and is designed to and is capable of precisely puncturing the cardiac interatrial septum without the use of a needle or other sharp instrument.

### RELATED APPLICATIONS

The present invention claims priority on and from U.S. Provisional Application Ser. No. 62/538,552, filed Jul. 28, 2017 and Entitled “Directional Balloon Transseptal Insertion Device for Medical Procedures” and U.S. Provisional Application Ser. No. 62/592,061, filed Nov. 29, 2017 and Entitled “Directional Balloon Transseptal Insertion Device for Medical Procedures,” both of which are incorporated by reference in their entirety.

### FIELD

The present invention relates generally to cardiac catheters, and more particularly, to a transseptal insertion device which is suitable for facilitating quick and safe transseptal puncture and insertion of a catheter through a cardiac septum to provide access to the left atrium in implementation of a left atrial intervention.

### BACKGROUND

Cardiac catheterization is a medical procedure in which a long thin tube or catheter is inserted through an artery or vein into specific areas of the heart for diagnostic or therapeutic purposes. More specifically, cardiac chambers, vessels and valves may be catheterized.

Cardiac catheterization may be used in procedures such as coronary angiography and left ventricular angiography. Coronary angiography facilitates visualization of the coronary vessels and finding of potential blockages by taking X-ray images of a patient who has received a dye (contrast material) injection into a catheter previously injected in an artery. Left ventricular angiography enables examination of the left-sided heart chambers and the function of the left-sided valves of the heart, and may be combined with coronary angiography. Cardiac catheterization can also be used to measure pressures throughout the four chambers of the heart and evaluate pressure differences across the major heart valves. In further applications, cardiac catheterization can be used to estimate the cardiac output, or volume of blood pumped by the heart per minute.

Some medical procedures may require catheterization into the left atrium of the heart. For this purpose, to avoid having to place a catheter in the aorta, access to the left atrium is generally achieved by accessing the right atrium, puncturing the interatrial septum between the left and right atria of the heart, and threading the catheter through the septum and into the left atrium. Transseptal puncture must be carried out with extreme precision, as accidental puncturing of surrounding tissue may cause very serious damage to the heart. In addition, transseptal puncture may require complicated instruments which are not helpful in guaranteeing the precision of the puncture.

The use of devices available today present many challenges for doctors attempting to puncture the interatrial septum and perform cardiac catheterization. Locating the interatrial septum, properly placing the distal end of the puncturing device at the desired location of the septum, safely puncturing the interatrial septum, avoiding accidental punctures, and tracking and maneuvering the catheter post-puncture, are among the many challenges facing those performing cardiac catheterization today.

Accordingly, there is an established need for a device that is suitable for facilitating quick and safe transseptal puncturing to provide access to the left atrium in implementation of a left atrial intervention.

### SUMMARY

Embodiments described herein overcome the disadvantages of the prior art. Embodiments provide for a device that is suitable for facilitating quick and safe transseptal puncturing to provide access to the left atrium in implementation of a left atrial intervention.

These and other advantages may be provided, for example, by a transseptal insertion device which is suitable for facilitating precise and safe transseptal puncture of a cardiac interatrial septum. The transseptal insertion device includes a sheath that defines at least one lumen therein and has a distal end that is closest to the cardiac interatrial septum of a patient when the transseptal insertion device is in use and a proximal end that is external to the patent, a balloon that is connected to the distal end of the sheath, in which the balloon, when inflated and the transseptal insertion device is in use, overhangs and extends past the distal end of the sheath, preventing accidental puncturing of the cardiac interatrial septum and stabilizing the transseptal insertion device against fossa ovalis of the cardiac interatrial septum, and a dilator that is positioned within the at least one lumen when the transseptal insertion device is in use. The dilator has a distal end and is designed to and is capable of precisely puncturing the cardiac interatrial septum without the use of a needle or other sharp instrument.

Embodiments may also overcome the disadvantages of the prior art and provide numerous advantages by including various features, including a dilator designed to remain sub-planar to the overhanging portion of the balloon until the dilator is extended to puncture the cardiac interatrial septum, the diameter of the lumen at rest being less than the diameter of the dilator, the sheath being made from malleable material capable of accommodating larger diameter devices within the lumen, the dilator including a dilator seal that is greater in diameter than the lumen and provides a water-tight seal of the lumen while the dilator seal remains within the lumen, sheath including inflation ports communicatively coupling the lumen to the balloon so that inflation liquid may be passed through lumen into the balloon, inflating the balloon, and the dilator seal being located on the distal end of the dilator such that moving the distal end of the dilator out of the lumen so that the dilator seal is external to the distal end of the sheath un-seals the lumen and causes the inflation liquid to flow out of the balloon and through the inflation ports, deflating the balloon. The transseptal insertion device may also include an energy source, such as a radio-frequency (RF) energy source, external to the proximal end of the sheath and operatively connected to the distal end of the dilator to deliver energy to the distal end of the dilator. The dilator may be designed to and is capable of precisely puncturing the cardiac interatrial septum using the energy, such as RF energy delivered to the distal end of the dilator. The transseptal insertion device of may also include an energy source external to the proximal end of the sheath and operatively connected to the balloon to deliver energy to the balloon and an external stabilizer connected to the proximal end of the sheath that stabilizes the transseptal insertion device and substantially prevents unwanted movement of the transseptal insertion device.

These and other advantages may be provided, for example, by a method of precisely and safely transseptal puncturing a cardiac interatrial septum. The method may include inserting a transseptal insertion device into a right atrium of a patient's heart, inflating the balloon, in which the inflated balloon overhangs and extends past the distal end of the sheath and the dilator is sub-planar to the balloon overhang, preventing accidental puncturing of the cardiac interatrial septum, extending the distal end of the sheath so that the inflated balloon is positioned against the fossa ovalis of the cardiac interatrial septum at a desired puncture point, thereby stabilizing the transseptal insertion device against fossa ovalis, extending the distal end of the dilator past the balloon overhang, puncturing the cardiac interatrial septum with the dilator without the use of a needle or other sharp instrument. Puncturing the cardiac interatrial septum with the dilator may include applying RF energy through the dilator to the cardiac interatrial septum. Extending the distal end of the dilator past the balloon overhang may cause the balloon to deflate. The method may include extending the distal end of the sheath past the cardiac interatrial septum and re-inflating the balloon.

These and other advantages may be provided, for example, by a transseptal insertion device which is suitable for facilitating precise and safe transseptal puncture of a cardiac interatrial septum. The transseptal insertion device may include a sheath that defines a lumen therein and has a distal end that is closest to the cardiac interatrial septum of a patient when the transseptal insertion device is in use and a proximal end that is external to the patent, the sheathe including inflation ports near the distal end that enable inflation fluid in the lumen to exit and enter the sheath. The device may further include a balloon that is connected to the distal end of the sheath, in which the balloon, when inflated and the transseptal insertion device is in use, overhangs and extends past the distal end of the sheath, preventing accidental puncturing of the cardiac interatrial septum and stabilizing the transseptal insertion device against fossa ovalis of the cardiac interatrial septum and the balloon is positioned over the inflation ports of the sheath so that inflation fluid may exit the sheath and enter the balloon, inflating the balloon, and exit the balloon and enter the sheath, deflating the balloon. The device may further include a dilator that is positioned within the lumen when the transseptal insertion device is in use. The dilator has a distal end and includes a dilator seal at the distal end that occludes the lumen when the dilator is sub-planar to the balloon overhang so that the balloon remains inflated after inflation fluid is passed through the lumen into the balloon and automatically deflates when the distal end of the dilator is extended sufficiently past the distal end of the sheath so that the dilator seal does not occlude the lumen.

These and other advantages may also be provided, for example, by a transseptal insertion device which is suitable for facilitating precise and safe transseptal puncture of a cardiac interatrial septum. The transseptal insertion device includes a sheath that defines at least one lumen therein and has a distal end that is closest to the cardiac interatrial septum of a patient when the transseptal insertion device is in use and a proximal end that is external to the patent, a balloon that is connected to the distal end of the sheath and a dilator that is positioned within the at least one lumen when the transseptal insertion device is in use, in which the dilator has a distal end and is designed to and is capable of precisely puncturing the cardiac interatrial septum. The balloon, when inflated and the transseptal insertion device is in use, overhangs and extends past the distal end of the sheath, preventing accidental puncturing of the cardiac interatrial septum and stabilizing the transseptal insertion device against fossa ovalis of the cardiac interatrial septum and includes one or more ultrasound transceivers that emit and receive ultrasound waves, convert the ultrasound waves to electrical signals, and transmit the electrical signals to an external imaging device that produces images of the cardiac interatrial septum from the received signal. The one or more ultrasound transceivers may be located on the balloon surface or inside the balloon, may be oriented towards the cardiac interatrial septum when the balloon is fully inflated and the distal end of the sheath is oriented towards the cardiac interatrial septum, may be oriented perpendicular to the sheath when the balloon is deflated, may be configured in the shape of a cross so that one or more ultrasound transceivers are oriented towards the cardiac interatrial septum when the balloon is fully inflated and the distal end of the sheath is oriented towards the cardiac interatrial septum and perpendicular to the sheath, may be configured in the shape of a disc, may be connected to an external imaging device through a wire that runs via lumen in sheath or to an external imaging device via WiFi or other wireless connection.

These and other advantages may also be provided, for example, by a transseptal insertion device which is suitable for facilitating precise and safe transseptal puncture of a cardiac interatrial septum. The transseptal insertion device includes a sheath that defines at least one lumen therein and has a distal end that is closest to the cardiac interatrial septum of a patient when the transseptal insertion device is in use and a proximal end that is external to the patent, a balloon that is connected to the distal end of the sheath, in which the balloon, when inflated and the transseptal insertion device is in use, overhangs and extends past the distal end of the sheath, preventing accidental puncturing of the cardiac interatrial septum and stabilizing the transseptal insertion device against fossa ovalis of the cardiac interatrial septum, and a dilator that is positioned within the at least one lumen when the transseptal insertion device is in use, in which the dilator has a distal end and is designed to and is capable of precisely puncturing the cardiac interatrial septum. The sheath includes one or more ultrasound transceivers that emit and receive ultrasound waves, convert the ultrasound waves to electrical signals, and transmit the electrical signals to an external imaging device that produces images of the cardiac interatrial septum from the received signals. The ultrasound transceivers may be oriented towards the distal end of the sheath or towards the proximal end of the sheath.

These and other advantages may also be provided, for example, by a transseptal insertion device which is suitable for facilitating precise and safe transseptal puncture of a cardiac interatrial septum. The transseptal insertion device includes a sheath that defines at least one lumen therein and has a distal end that is closest to the cardiac interatrial septum of a patient when the transseptal insertion device is in use and a proximal end that is external to the patent, a balloon that is connected to the distal end of the sheath, and a dilator that is positioned within the at least one lumen when the transseptal insertion device is in use, in which the dilator has a distal end and is designed to and is capable of precisely puncturing the cardiac interatrial septum. The balloon, when inflated and the transseptal insertion device is in use, overhangs and extends past the distal end of the sheath, preventing accidental puncturing of the cardiac interatrial septum and stabilizing the transseptal insertion device against fossa ovalis of the cardiac interatrial septum and is constructed of compliant and non-compliant material so that different portions of the balloon will expand differentially when the balloon is inflated. The transseptal insertion device may also include one or more ultrasound transceivers that emit and receive ultrasound waves, convert the ultrasound waves to electrical signals, and transmit the electrical signals to an external imaging device that produces images of the cardiac interatrial septum from the received signals.

These and other advantages may also be provided, for example, by a transseptal insertion device which is suitable for facilitating precise and safe transseptal puncture of a cardiac interatrial septum. The transseptal insertion device includes a sheath that defines at least one lumen therein and has a distal end that is closest to the cardiac interatrial septum of a patient when the transseptal insertion device is in use and a proximal end that is external to the patent, a balloon that is connected to the distal end of the sheath, in which the balloon, when inflated and the transseptal insertion device is in use, overhangs and extends past the distal end of the sheath, preventing accidental puncturing of the cardiac interatrial septum and stabilizing the transseptal insertion device against fossa ovalis of the cardiac interatrial septum, a dilator that is positioned within the at least one lumen when the transseptal insertion device is in use, in which the dilator has a distal end and a lumen defined therein, and a needle with a blunt end that is positioned within the lumen of the dilator and is designed to and is capable of precisely puncturing the cardiac interatrial septum with application of energy transmitted through the needle, in which the needle includes malleable and rigid portions. The needle may include a distal end and proximal end of the needle. The energy applied by the needle may be radio-frequency (RF) energy.

These and other objects, features, and advantages of embodiments of the present invention will become more readily apparent from the attached drawings and the detailed description, which follow.

### BRIEF DESCRIPTION OF THE DRAWINGS

The preferred embodiments described herein and illustrated by the drawings hereinafter be to illustrate and not to limit the invention, where like designations denote like elements, and in which:

FIG. 1A is a front perspective, cross-sectional view of an embodiment of a transseptal insertion device.

FIG. 1B is a front perspective, cross-sectional view of an embodiment of a transseptal insertion device showing a dilator extending partially through and extending out from device.

FIG. 1C is a front perspective, cross-sectional view of an embodiment of a transseptal insertion device showing a dilator extending partially through the device.

FIG. 2 is a front perspective, cross-sectional view of an embodiment of a transseptal insertion device showing inflated overhanging balloon and dilator positioned within device and subplanar to overhanging balloon.

FIG. 3 is a cross-sectional, end view of an embodiment of a transseptal insertion device and dilator shown prior to puncturing an interatrial cardiac septum with inflated overhanging balloon.

FIG. 4 is a front perspective, cross-sectional view of an embodiment of a transseptal insertion device with dilator advanced forward in order to tent an interatrial septum.

FIG. 5 is a front perspective, cross-sectional view of an embodiment of a transseptal insertion device with a transseptal wire advanced post-puncture through interatrial septum.

FIGS. 6A-6C are front perspective, cross-sectional views of an embodiment of a flexible transseptal insertion device with different angulations

FIG. 7 is a front perspective, cross-sectional view of an embodiment of a transseptal insertion device with radiofrequency energy capability.

FIG. 8 is a side view of an embodiment of transseptal insertion device with an overhanging balloon with marking.

FIG. 9 is a side view of an embodiment of transseptal insertion device with an overhanging balloon with a marker band.

FIG. 10 is a cross-sectional side view of an embodiment of a transseptal insertion device that includes a dilator with an electrode tip

FIG. 11A is a side view of an embodiment of a dilator that may be used in embodiments of a transseptal insertion device.

FIG. 11B is a side view of a distal end of an embodiment of a dilator that may be used in embodiments of a transseptal insertion device.

FIGS. 12A and 12B are side views of an embodiment of a transseptal insertion device, and interatrial septum, that includes a dilator with an ablation tip.

FIG. 13 is a side view of an embodiment of a transseptal insertion device with mechanical deflection capability.

FIG. 14 is side views of embodiments of curved dilators that may be used in embodiments of a transseptal insertion device.

FIG. 15 is a cross-sectional, side view of an embodiment of a steerable transseptal insertion device.

FIG. 16 is a perspective side view of a proximal end of an embodiment of a transseptal insertion device showing a handle and a stabilizer.

FIGS. 17A and 17B are side views of an embodiment of a transseptal insertion device with balloons capable of differential inflation.

FIG. 18 is a side view of a malleable or flexible transseptal needle that may be used in embodiments of a flexible transseptal insertion device with multiple angulations.

FIGS. 19A-19B are side views of embodiments of transseptal insertion device with ultrasound imaging or visualizing capability.

### DETAILED DESCRIPTION

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Embodiments of the present invention are directed to a transseptal insertion device or catheter which is suitable for facilitating quick and safe transseptal insertion of a needle or catheter through an interatrial cardiac septum/septal wall to provide access to the left atrium in implementation of a left atrial intervention. The transseptal insertion device is elongated yet has a relatively reduced length, and can be easily and safely turned within an atrium of the heart to achieve a correct orientation towards the cardiac septum.

In a first implementation of the invention, an embodiment includes a transseptal insertion device which is suitable for facilitating a precise and safe transseptal insertion of a needle, wire, dilator or catheter through a cardiac septum. The transseptal insertion device includes an overhanging balloon that, e.g., measures 2-12 mm and is either air or fluid filled when inflated. The dilator is sub-planar to the balloon at the time of contact with the atrial tissue (e.g., the interatrial septum).

In operation of an embodiment, once the balloon is noted to tent the interatrial septum, the dilator is advanced to a pre-specified position which is controlled by the movement of the dilator, which results in further tenting of the septum. Once it is confirmed that the tenting of the interatrial septum is accurate, the transseptal needle may be advanced across the septum. Following this the balloon may be deflated or may be kept inflated for more stability and for purpose of preventing the sheath from advancing too deep into the left atrium.

In another embodiment, the transseptal insertion device may include multiple dilators that may be inserted into the transseptal insertion device sheath. Each dilator may be pre-configured at a different angle so that, when inserted at in the sheath, the dilator will cause the transseptal insertion device to bend or flex so that the angle of a dilator to the interatrial septum to vary from 0-270°. This allows the transseptal insertion device to target different areas of the left atrium and parts of the heart accessible through the left atrium (e.g., the pulmonary veins, left atrial appendage, mitral valve, or the left ventricle). Each of these different areas of parts may require a different puncture/insertion point through the septum wall and a different angle of puncture and angle of approach in the left atrium. The pre-configured dilators enable the desired angulation to be chosen and effectuated.

Another embodiment of the transseptal insertion device may include an actuator which allows flexion and extension of the catheter and the transseptal sheath, which would, therefore, allow for motion of the transseptal sheath in all three-dimensional planes (i.e., x, y, z) and in both directions in each plane (e.g., up and down, left and right, etc.). In embodiments, the transseptal sheath used may have a variety of diameters or gauges. For example, the French (Fr) size or gauge of the transseptal sheath may range from 6-40 Fr in embodiments. The different gauge allows different size devices to be inserted through the transseptal insertion device.

Additionally, the dilator tip may house a cap, crown, or electrode tip that is connected to a thermal, laser, sonic, electrical, or radiofrequency energy source via the dilator at an exterior hub connected to the dilator through the proximal end of the transseptal insertion device. Accordingly, the dilator tip may deliver energy to the septum to create a safe and controlled puncture. In this manner, embodiments avoid the need to use needles and the disadvantages inhered therein. As noted, the types of energy delivered could be heat, cold, laser, sonic, electrical, or radiofrequency.

In an embodiment that includes a sheath dilator capable of transmitting energy, there is no need for a sharp, metallic transseptal needle. The dilator itself therefore may be moved across the interatrial septum and the lumen of the dilator then be used to pass a variety of wires or other tools into the left atrium. The lumen of the dilator may also be used for pressure monitoring and assessment of blood oxygen saturation through various sensors deployed in the lumen. For example, various devices may be attached to the dilator through an external hub located at the proximal end of the transseptal insertion device. Such devices may include a fluid-filled pressure transducer to measure pressure, e.g., in the atrial chamber, a solid-state sensor for measuring pressure, oximetry, or other characteristics of the atrial chamber, and/or a device for drawing fluid (e.g., blood) from the atrium for testing.

In an embodiment in which the dilator carries energy, the dilator may include a wire that may be energized by the dilator. The wire may be advanced into the left atrium to deliver energy to puncture the left atrial septum. The wire may be, for example, a wire with a 0.0014 or larger diameter. Such wires would significantly decrease the footprint of the device entering the left atrium.

In an embodiment, the sheath balloon is inflated using air or fluid via the sheath and there is no separate port or hypotube for inflation or deflation of the balloon. Such embodiments may include inflation ports in the sheath and a dilator seal on the dilator. The inflation ports may be implemented as multiple holes in the transseptal insertion device sheath that permit inflation fluid or gas to flow through a lumen in the sheath to the balloon and back out again when unsealed by the moving dilator seal

The dilator seal may be a bump or ridge located near the distal end of the dilator. When the dilator seal is covered by the overhanging sheath balloon, the seal is closed and the balloon may remain inflated. As the dilator is advanced beyond the overhanging balloon, the dilator seal moves beyond the seal zone, the dilator seal is opened or unsealed, resulting in the balloon automatically deflating. Prior art mechanisms require multiple parts on two different components that move relative to one another to seal and unseal an inflation chamber. The present embodiment requires only the ridge on the dilator.

When the balloon deflates, the inflation liquid or gas, which is inert, flows out of the balloon and into the heart, through which it is absorbed into the blood. The inflation air or fluid may include contrast agents enabling easier detection of inflation and deflation using imaging.

Embodiments may include radiopaque and echogenic markers on the balloon and the dilator. These markers may be in the form of letters, such as an E or a C. These markers allow for the appropriate positioning of the catheter in the 3-dimensional space (e.g., of the atrium) using imaging to view the markers and, therefore, the position of the balloon and dilator.

Embodiments may include a ring or band in the middle of the balloon. The ring in the middle of the balloon is for same purpose as the letter markings above—namely, to act as a navigational guide.

Various embodiments as described above are illustrated by the drawings. Shown throughout the drawings, embodiments of the present invention is directed toward a transseptal insertion device which is suitable for facilitating quick and safe transseptal puncturing of an interatrial septum and insertion of a catheter there thru to provide access to the left atrium in implementation of a left atrial intervention. Embodiments are suitable for other uses as well. Referring to the drawings described below, exemplary embodiments of a transseptal insertion device are illustrated. Together, the drawings illustrate method of using transseptal insertion device to puncture interatrial cardiac septum and extend and insert catheter (or other component inserted through transseptal insertion device) across interatrial septum and into left atrium. As shown, the transseptal insertion device is generally elongated and arranged along a longitudinal axis.

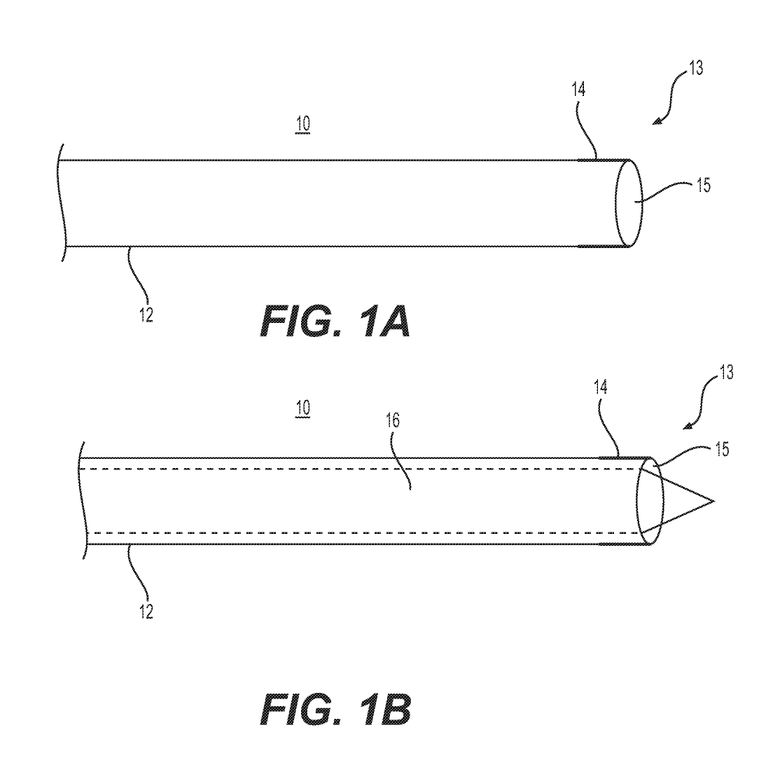

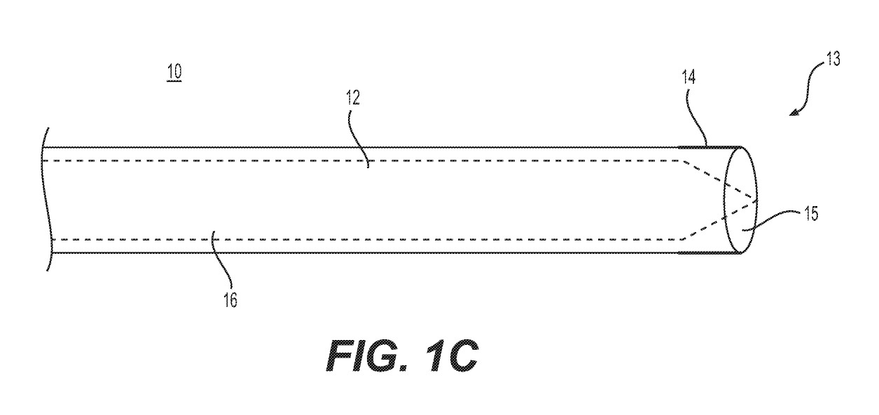

With reference now to FIGS. 1A-1C, shown is an embodiment of transseptal insertion device or catheter 10. Shown is the distal end of transseptal insertion device 10, i.e., the end of device 10 with opening through which dilator, catheter, and needle may extend, e.g., to puncture interatrial cardiac septum. As shown in FIG. 1A, transseptal insertion device 10 includes outer sheath or balloon shaft 12 and balloon 14 located at distal tip 13 of transseptal insertion device 10. Sheath 12 may contain and define a center lumen 15. Sheath 12 may be fabricated from various materials, including, e.g., polymers, including thermoplastics elastomers (TPEs) such as PEBA (e.g., Pebax®), nylons, thermoplastic polyurethanes (TPUs) such as Pellathane®, similar materials and combinations thereof. Sheath 12 may be referred to as catheter and used in cardiac catheterizations. After puncture, sheath 12 may be inserted through septum into left atrium. Alternatively, sheath 12 may contain a separate catheter that is inserted through septum post puncture.

Transseptal insertion device 10 also includes dilator 16, positioned in center lumen 15, as shown in FIG. 1B. Balloon 14 is preferably sealed, air-tight and water-tight, on both its ends to sheath 12, with openings or one or more inflation holes (not shown) in balloon 14 that open to lumen 15. Connected to sheath 12 as such, inflation liquid or gas may be passed through inflation holes into balloon 14, inflating balloon 14. Lumen 15 is kept sealed by dilator seal (not shown) that forms air and water tight seal of lumen 15 (until dilator seal on dilator 16 passes distal tip 13 and exits lumen 15).

With continuing reference to FIG. 1A, in view shown, overhanging balloon 14 is uninflated. Although cross-section of balloon 14 is shown on top and bottom of distal tip 13, balloon 14 preferably extends around circumference of distal tip or end 13 of transseptal insertion device 10 (see FIG. 4). Overhanging balloon 14 is of form such that balloon 14 overhangs or extends from distal tip 13 of sheath 12 when inflated.

In FIG. 1B, dilator 16 is shown positioned within and partially extending out of sheath 12, past distal tip 13 of device 10. Overhanging balloon 14 is uninflated and dilator 16 extends past balloon 14. It is noted that the relative sizes of sheath 12 and dilator 14 shown are for illustrative purposes as the diameter of dilator 14 may be relatively larger or smaller than shown in relation to the diameter of sheath 12, although dilator 14 necessarily has a smaller diameter than sheath 12. Although dilator 14 is shown to have a pointed end, dilator 14 may have a rounded or relatively flat end. Embodiments, as described herein, are designed and intended to puncture septum without use of a needle or other sharp instrument.

With reference now to FIG. 1C, dilator 16 is shown positioned within center lumen 15 of sheath 12. Tip of dilator 16 is positioned within distal tip 13 of transseptal insertion device 10 sub-planar to end of transseptal insertion device 10. The position shown is position dilator 16 may be in immediately prior to inflation of balloon 14. It is noted that the relative sizes of catheter/sheath 12 and dilator 16 shown are for illustrative purposes as the diameter of dilator 16 may be relatively larger or smaller than shown in relation to the diameter of sheath 12. Ordinarily, dilator 16 has smaller diameter or gauge then catheter/sheath 12, although fit of dilator 16 in catheter/sheath 12 is preferably snug enough so that dilator 16 does not move (laterally or axially) relative to position or “wobble” within transseptal insertion device 10. catheter 18 necessarily has a smaller diameter than sheath 12. In embodiments, sheath 12 material may be sufficiently malleable to enable larger diameter dilators 16, and other larger diameter devices, to be passed through sheath 12. In such embodiments, the sheath 12 will stretch to accommodate the larger diameter dilator 16 or other device.

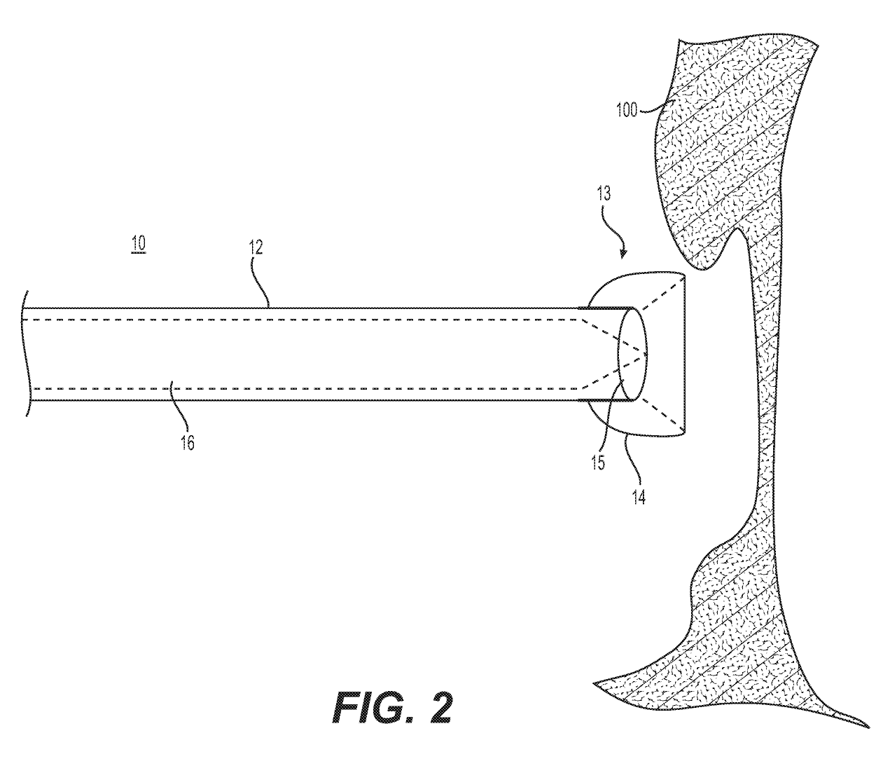

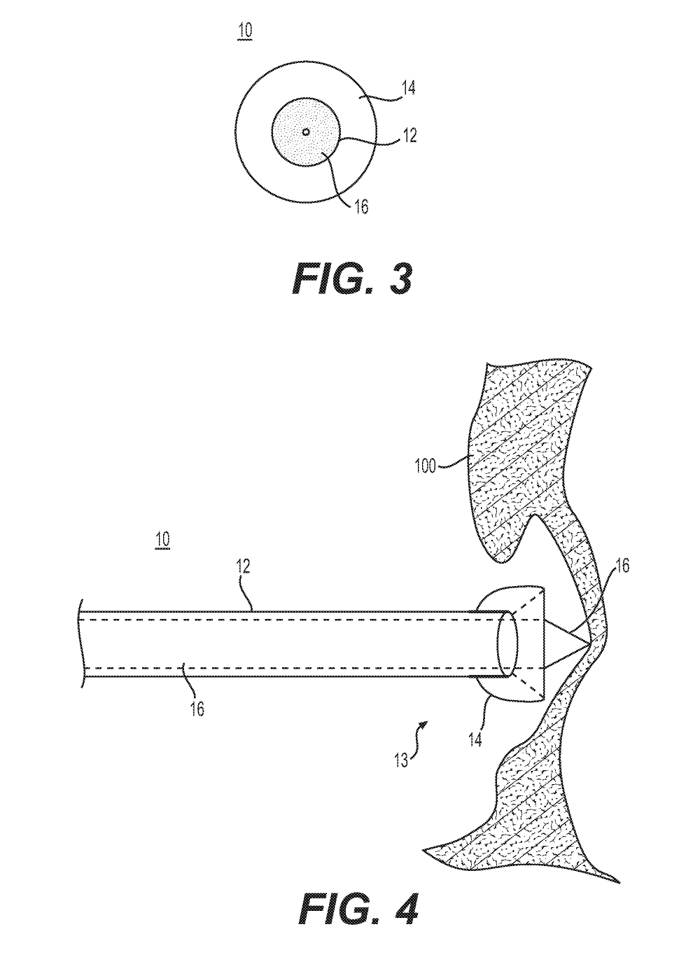

With reference now to FIG. 2, shown is distal end of an embodiment of transseptal insertion device 10 in which overhanging balloon 14 is inflated. Dilator 16 is shown positioned within center lumen 15 of sheath 12 with tip of dilator 16 positioned at distal tip 13 of transseptal insertion device 10 and sub-planar to overhanging balloon 14. The plane that is referred to here is the plane X-X, perpendicular to the axis of transseptal insertion device 10 and dilator 16, formed by the end of overhanging balloon 14. Hence, dilator 16 remains sub-planar to overhanging balloon 14 until operator intends balloon 14 to be deflated and dilator 16 to tent and puncture interatrial septum 100. As noted above, balloon 14 preferably extends completely around circumference of tip 13 of transseptal insertion device 10. Accordingly, FIG. 3 only illustrates cross-section of inflated balloon 14.

With reference now to FIG. 3, shown is a perspective, cross-sectional view of distal end an embodiment of transseptal insertion device 10 in which overhanging balloon 14 is inflated. As shown, inflated overhanging balloon 14 preferably extends around entire circumference of sheath 12 (and, therefore, device 10). Shown situated within lumen 15 of sheath 12 is tip of dilator 16. Tip of dilator 16 is positioned within tip 13 of transseptal insertion device 10, as it would be prior to being extended past tip 13 and puncturing an interatrial cardiac septum.

With reference now to FIG. 4, shown is shown is distal end of an embodiment of transseptal insertion device 10 with dilator 16 advanced forward in order to tent the interatrial septum 100. Dilator 16 is shown extending through center lumen 15 of sheath 12 and past overhanging balloon 14. Since dilator 16 is no longer sub-planar to overhanging balloon 14 and has moved past overhanging balloon 14, dilator seal (not shown) has moved and unsealed balloon 14, causing it to begin to deflate. Extended as such, and pressed against interatrial septum 100, dilator 16 tents the interatrial septum 100 away from transseptal insertion device 10.

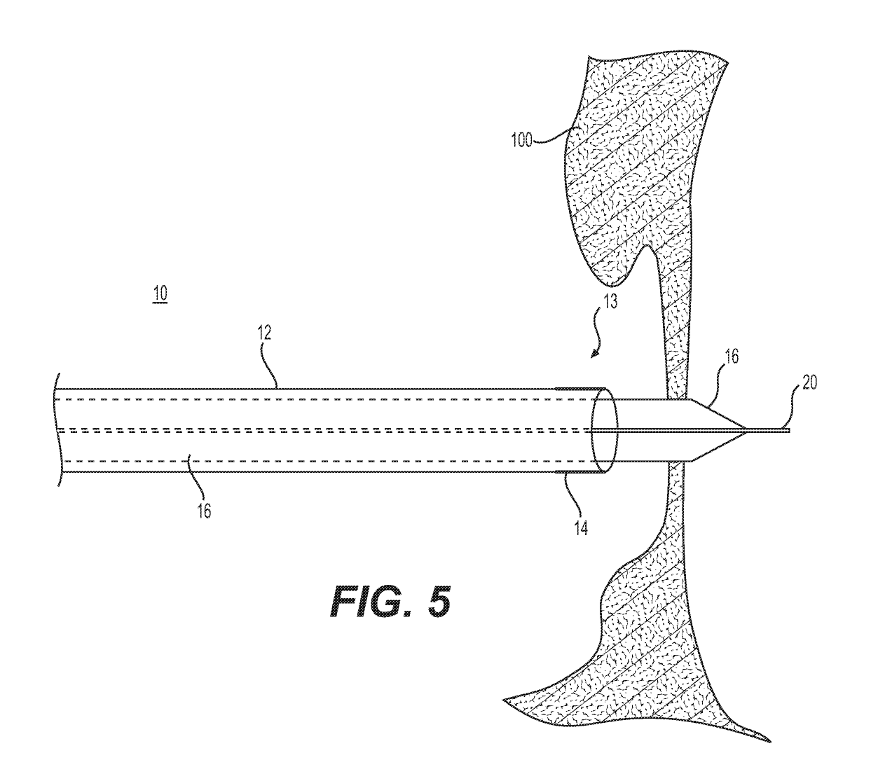

With reference now to FIG. 5, shown is shown is distal end of an embodiment of transseptal insertion device 10 with dilator 16 advanced forward through interatrial septum 100, after puncturing septal wall (e.g., through application of energy through dilator 16 as described herein) and transseptal wire or wire rail 20 extending through dilator 16 and into left atrium chamber 110. Wire rail 20 may sit in lumen 19 of dilator 16. Dilator 16 may be used as a conduit to advance the wire rail 20 into the left atrium.

Wire rail 20 may act as a guide for devices to enter the left atrium through the puncture in the septal wall made by transseptal insertion device 10. For example, wire rail may guide transseptal insertion device 10 or other catheters in the left atrium. In this manner, catheters may be advanced safely into the left atrium over or guided by wire rail 20. In an embodiment, wire rail 20 may be energized (e.g., to ablate or puncture the septum with energy delivered from source at proximal end of transseptal insertion device 10).

With continued reference to FIG. 5, dilator 16 preferably defines and includes an opening or lumen 19 extending through its tip and through which transseptal wire 20 extends. With dilator 16 extended as shown and tenting interatrial septum, septum may be punctured by energy delivered through cap or electrode at tip of dilator 16 (see below) and transseptal wire rail 20 extended through opening in tip of dilator 16 and through puncture made in interatrial septum by dilator 16 cap.

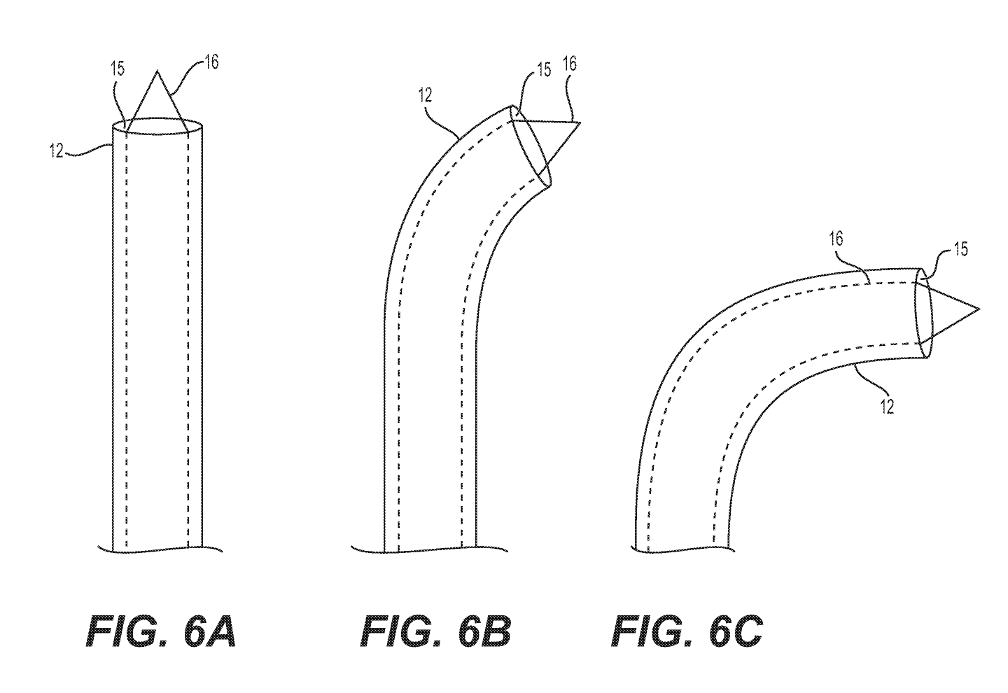

With reference to FIGS. 6A-6C, shown are different views of an embodiment of transseptal insertion device 10 with a flexible sheath 12 flexed or angulated at different angles. Transseptal insertion device 10 may be flexed or angulated depending on the anatomy of the atria using fixed angled dilators 16 that are inserted into lumen 15 of sheath 12, causing sheath 12 to flex. Such fixed angled dilators 16 may be, e.g., any angle from 0-270°. Alternatively, sheath 12 and dilator 16 may be both flexible (preferably, needle and catheter inserted through such flexible sheath 12 are flexible or malleable, at least in part) and transseptal insertion device 10 may be flexed or angulated, thereby flexing or angulating sheath 12 and dilator 16, using, e.g., a handle or wire (not shown) connected to tip 13 of device 10. Handle and/or wire may also be used to turn or flex or move tip 13 of transseptal insertion device 10, e.g., moving tip 13 of sheath “up” or “down” or “left” or “right” or angulating tip 13 relative to axis of sheath 12 as shown.

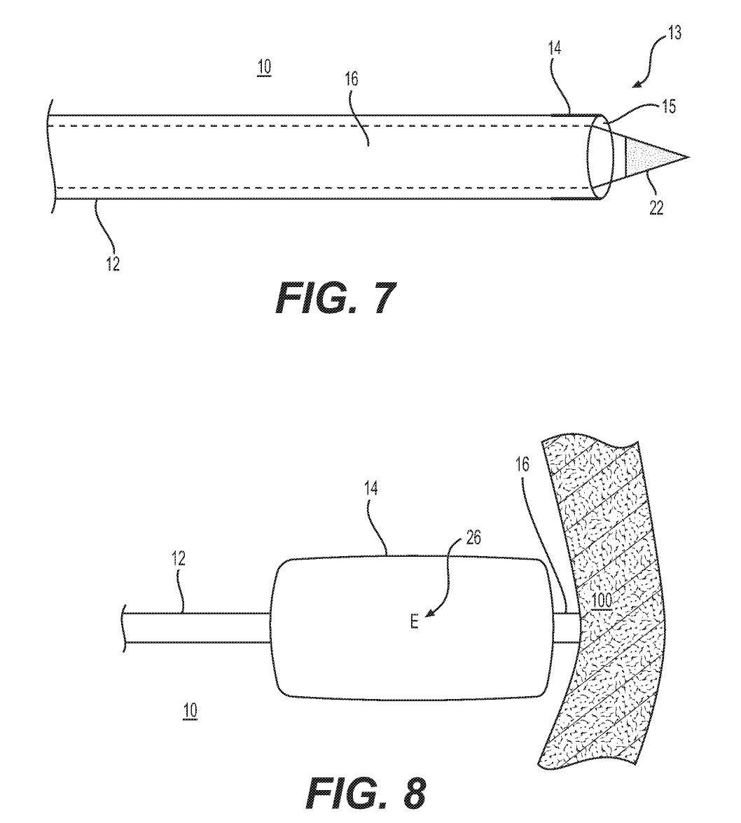

With reference now to FIG. 7, shown is an embodiment of transseptal insertion device 10 with radiofrequency energy capability. Transseptal insertion device 10 shown includes sheath 12, overhanging balloon 14, and dilator 16. Dilator 16 may include cap or crown 22, on distal end as shown, with RF energy capability or capable of delivering RF energy. Alternatively, cap or crown may include or be an RF electrode. Dilator 16 may be connected, e.g., on proximate end (not shown) to a radiofrequency (RF) energy source (not shown) at, e.g., external hub, that provides RF energy to cap or crown 22. The RF energy may be delivered through dilator 16. So equipped with cap or crown 22, dilator 16 may tent interaxial septum and create puncture of interaxial septum through delivery of RF energy. In this embodiment, the use of a sharp needle may be avoided.

As described above, embodiments may be capable of delivering other energy, such as thermal, laser, sonic, or electrical energy for the purposes of puncturing the septum. Such embodiments may be constructed in a similar manner, with dilator or needle including cap or crown at a distal end capable of delivering thermal, laser, sonic, or electrical energy, and such energy may be delivered through dilator or needle connected, e.g., on proximate end (not shown) to a thermal, laser, sonic, or electrical energy source (not shown) at, e.g., external hub. So connected, dilator or needle may use thermal, laser, ultrasound, or electrical energy to puncture interaxial septum, avoiding the need for a sharp needle.

A significant challenge for operators of transseptal devices today is the difficulty in determining how posterior (towards the back of a patient) the transseptal device is located. The left atrium is on the posterior side of the heart. It is, therefore, often critical to be able to determine how posterior is the distal tip 13 of transseptal insertion device 10 in order to successfully locate the interatrial septum. Generally tracking the location of the distal end of transseptal insertion device 10 is critical to safe operation. With reference now to FIG. 8, shown is distal end of an embodiment of transseptal insertion device 10 with inflated overhanging balloon 14. Balloon 14 shown is an embodiment with one or more markers 24. Marker 24 may be, e.g., a radiopaque and/or echogenic marker 24. As a radiopaque or echogenic marker, marker 24 will be visible on scanners used by those performing cardiac catheterizations. The markers 24 may be in the form of letters, such as an E or a C. Marker 24 enables the appropriate positioning of balloon 14 and catheter 18 in the 3-dimensional space (e.g., of the atrium) using imaging to view the marker 24 and, therefore, the position of balloon 14.

Specifically, in operation, the less posterior distal tip 13 is positioned, the more of the E (or C) will be shown. As operator of transseptal insertion device 10 turns or rotates distal tip 13 toward posterior of patient, less of the arms of the E will be seen. In a preferred embodiment, when only the vertical portion of the E is visible (i.e., appearing as an I) distal tip 13 will be rotated to its maximum posterior position. Consequently

With continuing reference to FIG. 8, balloon 14 is shown as inflated. However, distal end of dilator 16 is shown extruding or extending distally from balloon 14, past plane formed by distal end of inflated balloon 14. According, dilator 16 has been moved into the tenting and puncturing position, adjacent to interaxial septum, dilator seal (not shown) has exited or soon will exit sheath 12, balloon 14 is deflating or will soon deflate, and puncture of the interaxial septum is imminent.

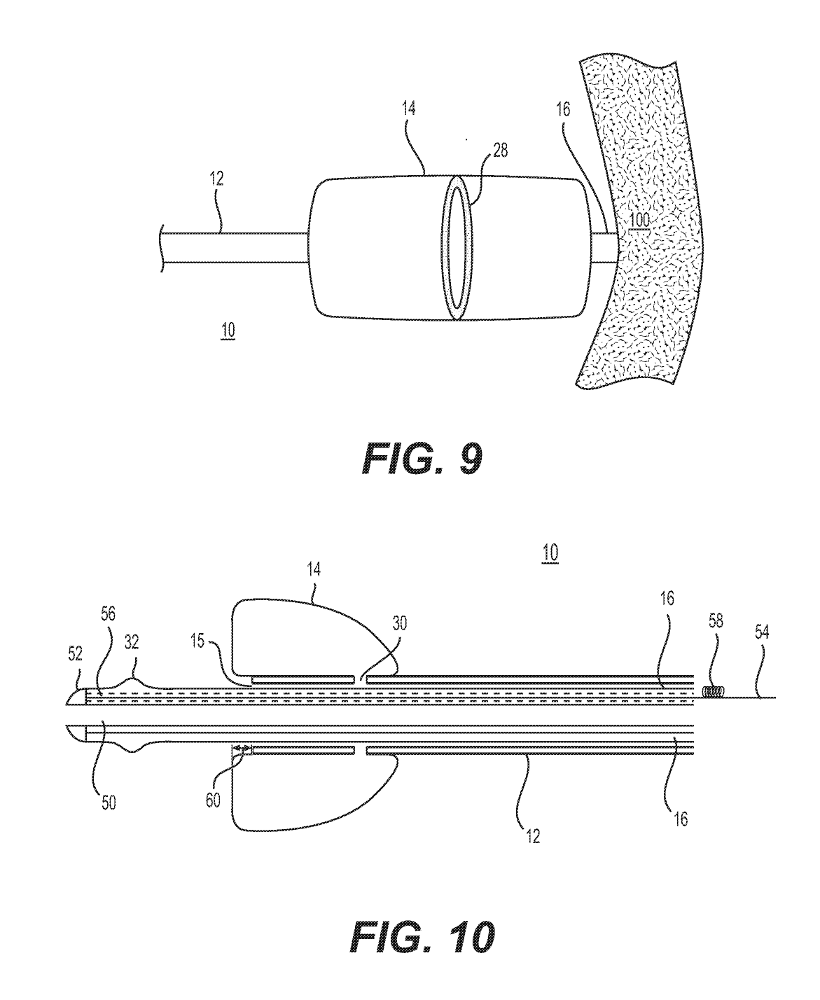

With reference now to FIG. 9, shown is another embodiment of overhanging balloon 14 which may be deployed in embodiments of transseptal insertion device 10. Overhanging balloon 14 may include ring or band 28 around a portion of balloon 14. Ring or band 28 may serve as a marker, similar to markers 24 shown in FIG. 8. Hence, ring 28 may be radiopaque or echogenic and may be view by scanning devices used for visualization in cardiac catheterizations (e.g., fluoroscopic imaging devices). Similar to the letter E or C, the view of the ring 28 changes as the distal tip 13 of transseptal insertion device 10 moves more posterior. When in a least posterior position, ring 28 may appear as just a line or band positioned across axis of transseptal insertion device 10. When device 10 is rotated so that distal tip 13 is significantly closer to the posterior, ring 28 may appear as a full “flat” circle or ring. In FIG. 8, distal tip 13 is partially rotated so that ring 28 is partially visible.

With reference to both FIGS. 8 and 9, the marker 24 and ring 28 are described and shown as located on balloon 14. In embodiments, marker 24 and/or ring 28 may also be located on sheath 12 and/or dilator 16. So located, marker 24 and/or ring 28 would operate in effectively the same manner as described above (i.e., the arms of the E would disappear as the distal end was moved more to the posterior and the ring would become more visible). Markers 24 and/or rings 28 may be placed on all three of balloon 14, sheath 12, and dilator 16, or a combination thereof.

With reference now to FIG. 10, shown is distal end of an embodiment of transseptal insertion device 10 that includes dilator 16 with electrode tip. Shaft of dilator 16 defines and contains a center lumen 50. Lumen 50 may be defined in the range of, but not limited to, 0.020″-0.040″. Dilator 16 may be made from a polymer material (e.g., HDPE, LDPE, PTFE, or combination thereof). Dilator shaft 16 shown includes a distal electrode tip 52. Electrode tip 52 may be comprise a metallic alloy (e.g., PtIr, Au, or combination thereof). In preferred embodiments, the size and shape of electrode tip 52 is selected to be sufficient to generate a plasma for in vivo ablation of tissue in an applied power range of, but not limited to, 20-30 W. Electrical conductor 54 extends from electrode tip 52 to the proximal end (not shown) of the dilator 16. Electrical conductor 54 may run axially through an additional lumen 56 defined by and contained in dilator shaft 16. Electrical conductor 54 may contain a coil feature 58 to accommodate lengthening during bending or flexing of dilator 16.

Dilator 16 may also include a distal dilator seal 32 for occlusion of sheath shaft 12 center lumen 15. Dilator seal 32 may be a ring that extends around entire circumference of dilator 16. Dilator seal 32 may be in the range of, but not limited to, 0.000″-0.005″ larger in diameter than center lumen 15. Distal seal 32 occludes lumen 15, forming a liquid and gas tight seal so that balloon 14 remains inflated, until distal seal 32 exits lumen 15. Attached to distal end of sheath 12 is contains overhanging balloon 14. Overhanging balloon 14 may be made from a polymer material (e.g., PET, Nylon, Polyurethane, Polyamide, or combination thereof). Overhanging balloon 14 may be in the range of, but not limited to, 5-20 mm in diameter and 20-30 mm in length. Overhanging balloon 14 may be inflated via injection of fluid (or gas) from the proximal end of sheath 12 center lumen 15 while distal dilator seal 32 occludes center lumen 15 distal to inflation ports 30 in sheath 12. Inflation ports 30 are preferably defined flush to surface of sheath 12 and communicate with lumen 15 (e.g., inflation ports may simply be holes defined in sheath that connect lumen to exterior of sheath). Balloon 14 is preferably connected to sheath 12 so that inflation ports 30 communicate with interior of balloon 14 and provide pathway for inflation fluid or gas to enter and inflate balloon 14 (and exit and deflate balloon 14). During the proper functioning or operation of transseptal insertion device 10 for puncturing the interatrial septum, balloon 14 is deflated when dilator 16 moves out of lumen 15 and dilator seal 32 moves distal and outside of sheath 12. However, deflation of overhanging balloon 14 may occur either via positioning of dilator seal 32 proximal to inflation ports 30 or distal and outside of sheath 12. Overhanging balloon 14 is of form such balloon 14 overhangs or extends from distal end 13 of sheath 12. Overhang or extension 60 may be in the range of, but not limited to, 0.0 mm-5.0 mm. The end of the overhang or extension 60 is the plane to which dilator 16 remains sub-planar until moving to tent and puncture the interatrial septum.

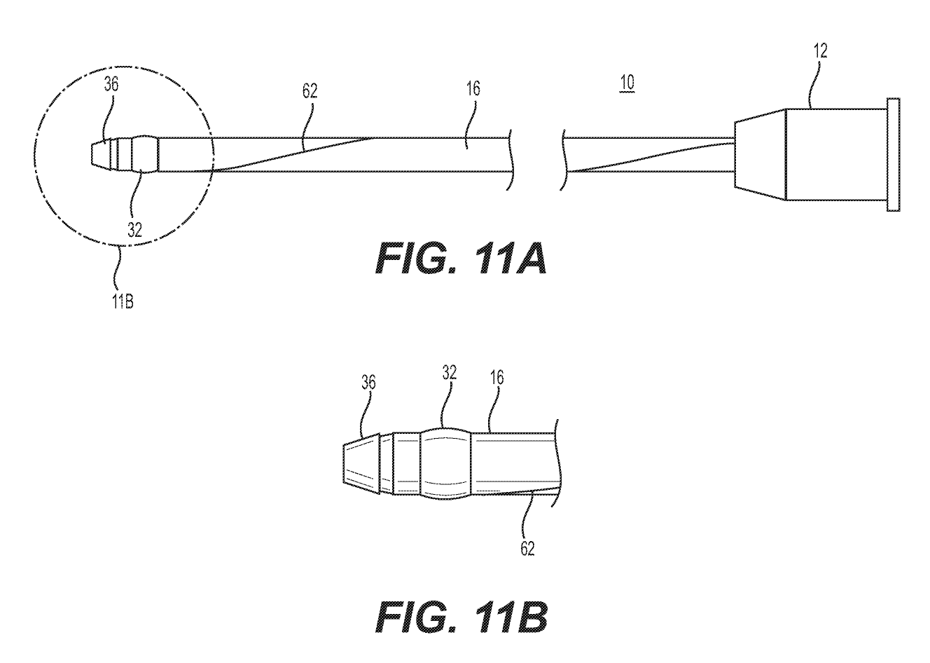

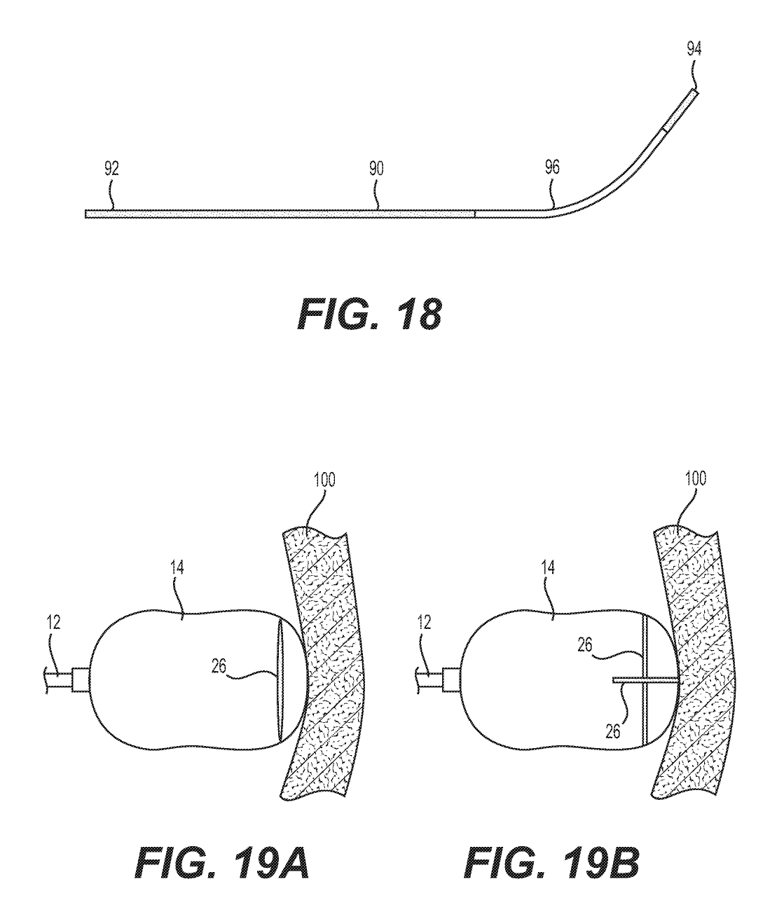

With reference now to FIGS. 11A and 11B, shown is a distal end of an embodiment of transseptal insertion device 10 with RF capability, with dilator 16 extended out from sheath 12 shaft. Dilator 16 includes a RF cap or tip 36 that may deliver RF energy for interatrial septum ablation purposes, as described above. RF cap 36 may be connected to RF energy source (not shown) at proximal end (not shown) of transseptal insertion device 10 with conductor 62. Conductor 62 may wrap around shaft of dilator 16 as shown. Alternatively, conductor 62 may be extend through a lumen (not shown) of dilator 16 (e.g., such as lumen 56 shown in FIG. 18). Dilator 16 may include distal dilator seal 32 for sealing center lumen 15 (not shown) of sheath 12 and inflation ports 30 (not shown).

Variations of the above embodiments are within the scope of the invention. For example, the dilator shaft may have a preformed shape other than straight. The dilator shaft may contain a deflection apparatus. The electrode tip may be the distal dilator seal. The electrical conductor may wrap around the center lumen. Sheath or balloon shaft may contain a deflection apparatus.

Embodiments of transseptal insertion device 10 can successfully assist surgeons in carrying out at least one of the following techniques: visualization and stabilization of the intra atrial septum; visualization and stabilization of the fossa ovalis; and, guidance for transseptal puncture and across septum into safe zone of left atrium (away from structures such as aorta).

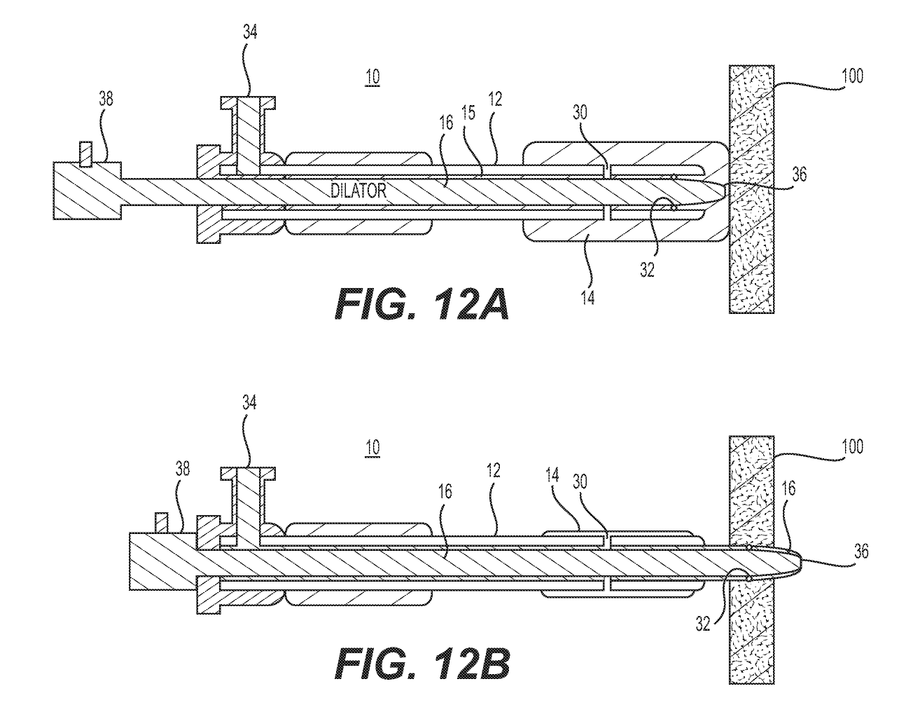

With reference now to FIGS. 12A-12B, shown is another embodiment of transseptal insertion device 10 that inflates overhanging balloon 14 using gas or fluid via sheath 12. Embodiments include no separate port or hypotube for inflation or deflation of balloon 14. Transseptal insertion device 10 may include inflation ports 30 in sheath 12 and dilator seal 32 on dilator 16. Gas (e.g., air) or liquid is input into sheath 12 through inlet or port 34. Gas or liquid exits sheath 12 through inflation ports 30, inflating balloon 14 until fully inflated or inflated as much as desired. When dilator seal 32 is covered by inflated, overhanging sheath balloon 14, as shown in FIG. 12A, dilator seal 32 is closed and balloon 14 remains inflated. As dilator 16 is advanced beyond overhanging balloon 14, as shown in FIG. 12B, dilator seal 32 moves beyond the seal zone, dilator seal 32 is opened or unsealed, resulting in balloon 14 automatically and rapidly releasing inflation gas or fluid and deflating. The inflation gas or liquid exits the balloon 14 and the lumen 15 as noted above and is absorbed by the body and bloodstream. As such, inflation gas or liquid is inert and non-harmful. Inflation gas or fluid may include contrast agents enabling easier detection of inflation and deflation using imaging.

Embodiments of transseptal insertion device 10 include ablation tip 36, e.g., radiofrequency (RF) ablation tip 36 (similar to crown or cap 22) that may be used to deliver RF or other energy to ablate interaxial septum (septal wall) in order to puncture and create opening in septum. Trans septal insertion device 10 may include energy source at proximal end to deliver RF or other energy through dilator to ablation tip 36. Energy source may be, e.g., RF ablation connector on external hub 38. Ablation connector on external hub 38 may be connected to proximal end of dilator 16, as shown.

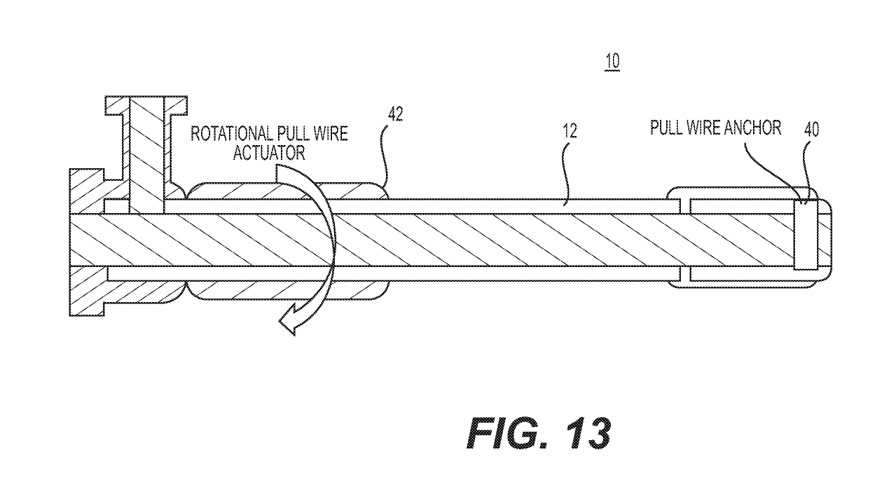

With reference now to FIG. 13, shown is an embodiment of transseptal insertion device 10 that includes a mechanical deflection mechanism. Mechanical deflection mechanism may enable distal end of sheath 12 to be deflected or angulated to various angles with respect to axis of transseptal insertion device 10. Mechanical deflection mechanism may include a pull wire anchor 40 affixed to distal end of sheath 12 and pull wire actuator 42 connected to pull wire anchor 40 with pull wire (not shown). Rotation of pull wire actuator 42, as shown, may exert force on pull wire anchor 40 that deflects or angulates distal end of sheath 12. Pull wire actuator 42 may be rotated by handle connected thereto (not shown). Deflection or angulation of distal end of sheath 12 may enable better intersection (e.g., more perpendicular, flush) with interaxial septum and, therefore, better puncture and insertion by transseptal insertion device 10.

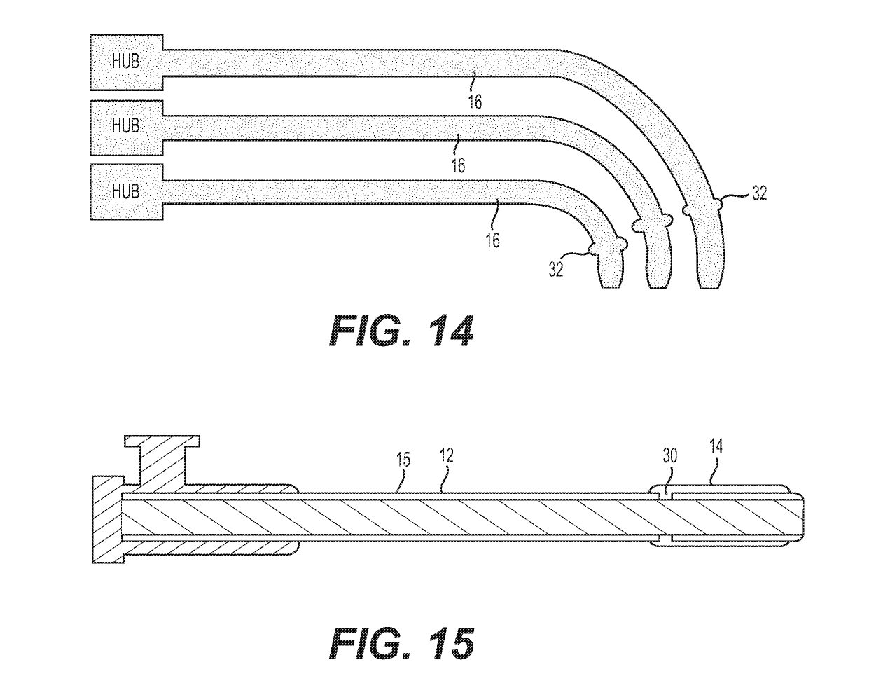

With reference now to FIG. 14, shown are three (3) embodiments of curved dilators 16, each with a different curve profile (i.e., different angle of deflection or curve). Curved dilators 16 may be used in embodiments of transseptal insertion device 10 with flexible or malleable sheath 12. Such a flexible or malleable sheath 12 may be referred to as a steerable sheath 12 as it is ‘steered” by curved dilator 16 inserted in sheath 12.

With reference now to FIG. 15, shown is an embodiment of transseptal insertion device 10 that includes a steerable sheath 12. In embodiment shown, distal end of sheath 12 is flexible or malleable so that sheath 12 may bend or angulate, i.e., be steered, when, for example, curved dilator 16 is inserted. Proximal body of sheath 12 may be stiffened so that curved dilator 16 may be more easily pushed through sheath 12 when inserted therein.

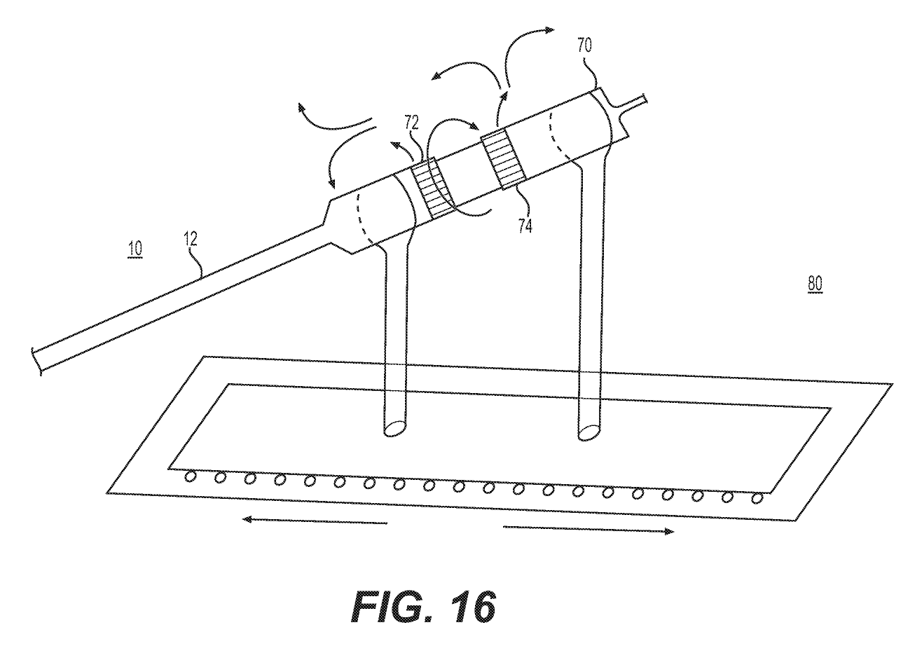

Embodiments of transseptal insertion device 10 may include a stabilizer in which the exterior of the catheter would be placed and which allows for very precise movements of the catheter. With reference now to FIG. 16, shown is an embodiment of transseptal insertion device/catheter 10 with an external stabilizer 80. Stabilizer 80 keeps proximal end of transseptal insertion device 10 stable while allowing movement of transseptal insertion device 10 towards the distal and proximal ends of device 10, rotational/torqueing movement of proximal end of device 10, and manipulation of dials or other controls of device 10. In effect, stabilizer 80 substantially prevents unwanted movement of the transseptal insertion device 10 and, importantly, distal end of sheath 12, balloon 14, and dilator 16.

Stabilizer 80 includes connecting rods or arms 82 that connect stabilizer 80 to handle 70 at proximal end of transseptal insertion device 10. Connecting arms 82 are attached to stabilizer platform 84. Connecting arms 82 preferably hold the handle 70 securely and tightly, while permitting desired rotational movements and control manipulation. Stabilizer platform 84 is moveably attached to stabilizer base 86 so that stabilizer platform 84, and hence handle 70 and transseptal insertion device 10, may be slid forwards and backwards along axis of transseptal insertion device 10 towards and away from insertion point in patient (typically femoral vein at the groin of patient). Stabilizer base 86 is typically secured to a flat, stable surface, such as a table, or the leg of the patient. Configured as such, stabilizer 86 prevents unwanted vertical, rotational, or other movement of transseptal insertion device 10 and its handle 70, keeping transseptal insertion device 10 and its handle 70 stable while permitting precise manipulation of handle 70 and its controls.

With continuing reference to FIG. 16, as shown, proximal end of transseptal insertion device 10 may include a handle 70 for control and manipulation of transseptal insertion device 10 and, particularly, dilator 16 and distal end of dilator 16. Handle 70 may include a dial 72 that may be used to turn or deflect distal end of dilator 16, effectively moving the distal end of dilator 16 up or down in relation to axis of transseptal insertion device 10 (as indicated by arrows in FIG. 16). Handle 70 may also include dial 74 for extruding/extending distal end of dilator 16 out of sheath 12 and retracting dilator 16 back into sheath 12, effectively moving dilator 16 along axis of transseptal insertion device 10 (as indicated by arrows in FIG. 16). Handle 70 may also be rotated, as indicated by rotational arrow in FIG. 16, in order to deflect or turn distal end of transseptal insertion device to left or right in relation to axis of transseptal insertion device 10, increasing or decreasing dilator 16 angle of deflection in that direction. If dial 72 moves distal end of dilator 16 along Y axis, and transseptal insertion device 10 axis is considered the Z axis, so that dial 74 moves dilator 16 along Z axis rotating handle 70 moves distal end of transseptal insertion device 10 (and hence distal end of dilator 16) along X axis. Handle 70 includes port 76 to sheath lumen 15 through which dilator 16 and other devices inserted into transseptal insertion device 10 may be inserted. Handle 70 may also include one or more tubes or other ports permitting connection to external hubs and external energy sources, inflation liquids or gas,

In embodiments shown herein, balloon 14 and dilator 16 may be used as energy sources in the left atrium and may be used to deliver energy to the pulmonary veins, left atrial appendage, mitral valve and the left ventricle present in the left atrium. Such embodiments may include external energy sources connected to balloon 14 and/or dilator 16 through wires or other conductors extending lumen in sheath 12. Delivery of energy via balloon 14 or dilator 16 may be thermal/Cryo or radiofrequency, laser or electrical. The delivery of such energy could be through a metallic platform such as a Nitinol cage inside or outside balloon 14. Transseptal insertion device 10 may also include an energy source external to the proximal end of the sheath and operatively connected to balloon 14 to deliver energy to balloon 14.

In other embodiments, balloon 14 may be re-inflated once distal tip 13 of transseptal insertion device 10 is passed through interatrial septum. Dilator 16 would be retracted into sheath 12 so that dilator seal 32 re-seals lumen 15 and inflation liquid or gas supplied through lumen 15 and into balloon 14. In this manner, balloon 14 may be used as occlusion balloon in the left atrium, including in the pulmonary veins and left atrial appendage. In this manner, balloon 14 may be used for performing occlusion procedures in the left atrium.

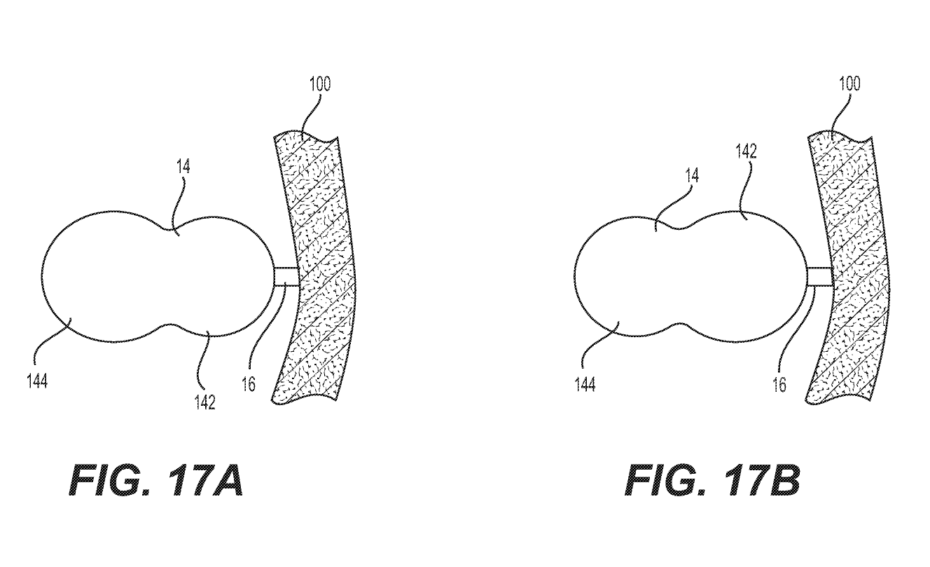

With reference now to FIGS. 17A and 17B shown is an embodiment of transseptal insertion device 10 enabling differential expansion of balloon 14. Differential expansion of balloon 14 enables balloon 14 inflation to be adjusted based on the needs of the device operator and the conditions present in the patient's heart. For example, the size of the fossa ovalis portion of the interatrial septum may dictate the desired size of the inflated balloon 14 needed at the puncture site (interatrial septum if often punctured through the fossa ovalis). Fossae can vary greatly in size. The larger the fossa, the harder it will be to tent the interatrial septum with balloon 14. Large fossa tend to be saggy and more difficult to manipulate. Hence, with a large fossa, a larger distal end of balloon 14 will make proper tenting of the interatrial septum easier. Indeed, it may be ideal to have balloon 14 inflated uniformly until intersecting or passing through fossa and then differentially expanding distal end 142 of balloon 14 to move fossa out of the way. In FIG. 17A, distal end or portion 142 of balloon 14 is smaller (less expanded) than proximal end 144 of balloon 14.

Oppositely, the smaller the fossa, the easier it will be to tent the interatrial septum but, there will be less room to maneuver balloon 14 near interatrial septum. Consequently, a smaller distal end of balloon 14 is desired. It also may be beneficial to expand the proximal portion 144 more in order to help fix or secure balloon 14 in place. In FIG. 17B, distal end or portion of balloon 14 is larger (more expanded) than proximal end or portion of balloon 14. In both FIGS. 17A and 17B, dilator 16 has extruded from sheath 12 and past distal end of balloon 14, tenting interatrial septum 100, and puncture is imminent.

This differential expansion of balloon 14 may be achieved, e.g., by using different materials for different portions of balloon 14 (e.g., a more expandable material for distal end 142 than proximal end or portion 144, or vice versa). In general, balloon 14 may be made of either compliant or non-compliant material, or a combination thereof. Compliant material will continue expanding as more inflating liquid or gas is added to balloon 14 (at least until failure). Non-compliant material will only inflate up to a set expansion or designated inflation level. Combinations of compliant and non-compliant material may be used to provide a differentially expanding balloon 14. For example, distal end 142 may be formed from compliant material and proximal end 144 from non-compliant material to enable a larger distal end 142. Oppositely, proximal end 144 may be formed from compliant material and distal end 142 from non-compliant material to enable a larger proximal end 144. Other means for providing differential expansion of balloon 14 may be used, such as applying energy to different portions of balloon 14 to increase or decrease the compliance, and expandability, of that portion.

Balloon 14 may also be used to direct other equipment into these anatomical locations or be used as an angiographic or hemodynamic monitoring balloon. Differential expansion of balloon 14 may be utilized for proper orientation or direction of such equipment.

With reference now to FIG. 18, shown is an embodiment of a malleable transseptal needle 90 that may be used with transseptal insertion device 10 with a flexible sheath or otherwise capable of multiple angulations. In embodiments, malleable transseptal needle 90 may be of a variety of diameters and lengths. For example, embodiments include an 18 gauge transseptal needle and that is available in 71 cm, 89 cm, and 98 cm lengths. In embodiments, the malleable transseptal needle 90 has different stiffness in a proximal segment 92, distal segment 94, and in a middle segment 96 between. For example, malleable transseptal needle 90 may be stiffer in the proximal segment 92 and distal segment 94 and more flexible (less stiff) in a middle segment or mid-section 96. The mid-section may be the section where the catheter 10 and dilator 16 angulate. In an embodiment, malleable transseptal needle 90 is used and a control handle provided that enables three-dimensional movements. Malleable transseptal needle 90 shown is, preferably, malleable or flexible at least in part. Proximal end 92 of malleable transseptal needle 90 may be stiff (e.g., made from a stiff material, such as a metal). Mid-section or middle 96 of malleable transseptal needle 90 may be malleable or flexible (e.g., made from a flexible, malleable material, such as rubber). Accordingly, mid-section may flex or bend, enabling malleable transseptal needle 90 to pass through angulated or flexed sheath 12.

Distal end 94 of malleable transseptal needle 90 (i.e., end that punctures interatrial cardiac septum) may be stiff with a cap or electrode at its tip for delivering energy to interatrial septum to puncture interatrial septum. In embodiments, transseptal needle is able to transmit radiofrequency energy to create a controlled septal puncture. Such a transseptal needle may or may not be malleable, but is able deliver RF energy through a cap or crown (e.g., an electrode) at its distal end tip. The needle 90 may be connected, e.g., on proximate end (not shown) to a radiofrequency (RF) energy source (not shown) at, e.g., external hub, that provides RF energy through needle to its distal end tip. In such an embodiment, dilator 16 may tent interaxial septum and RF energy capable transseptal needle may create puncture of interaxial septum through delivery of RF energy.

Embodiments of transseptal insertion device 10 may include a variety of additional features or components that improve its operation and enable increased control or additional functions. For example, the transseptal sheath 16 or balloon 14 may house (inside or on) an ultrasound chip or transducer which may be used to guide the insertion procedure. Ultrasound chip or transducer emits and receives ultrasound energy, that may be detected by known ultrasound visualization devices, to create an image of the cardiac chambers (e.g., the right atrium, fossa, interatrial septum, left atrium, atrial appendage, mitral valve, ventricle, etc.). Ultrasound chips and transducers are transducers that convert ultrasound waves to electrical signals and/or vice versa. Those that both transmit and receive may also be called ultrasound transceivers; many ultrasound sensors besides being sensors are indeed transceivers because they can both sense and transmit. Such imaging will allow the operator(s) of transseptal insertion device 10 to visualize the cardiac chambers and the determine the location of the distal end or tip 13 of transseptal insertion device 10, enabling more precise operation of transseptal insertion device 10. Such a ultrasound chips or transducers used may be similar to ultrasound chip or transducer described in US Pat. App. Pub. 2003/019546, which is herein incorporated by reference, or any other ultrasound transducer known to those of ordinary skill in the art that may be fabricated on scale small enough to be deployed on or in sheath 16 or balloon 14.

When deployed on balloon 14, ultrasound chip or transducer may be forward facing (towards the left atrial septum) and deployed in the shape of a disc, a cross, or another rendition. So deployed, ultrasound chip or transducer enables visualization of the interatrial septum and the left atrial structures. Such an ultrasound chip may be connected to an imaging system either via a cable or wire that runs via sheath 12 or dilator 16 or connected via WiFi or other wireless connection.

With reference now to FIGS. 19A-19B, shown are embodiments of transseptal insertion device 10 with ultrasound imaging or visualizing capability. Balloon 14 shown includes one or more ultrasound chips or transducers 26 deployed in or on balloon 14. Ultrasounds chips or transducers 26 may be ultrasound transceivers that both emit and receive waves, convert the ultrasound waves to electrical signals, transmit the electrical signals, e.g., through a wire that runs via sheath 12 or dilator 16 or connected via WiFi or other wireless connection, to an external imaging device that produces images from the received signals (both still and video images). Ultrasound chips or transducers 26 may be affixed to interior or exterior surface of balloon 14. Ultrasound chips or transducers 26 may be arranged in a line, disc, or cross-shape. Ultrasound chips or transducers 26 may be arranged to be forward facing (e.g., on distal end of balloon facing towards interatrial septum), as shown in FIG. 19A, or in a different direction/orientation, such as sideways and forward facing (e.g., facing towards interatrial septum and facing perpendicular to the distal or front end), as shown in FIG. 19B. Indeed, orientation of ultrasound chips or transducers 26 may depend on whether balloon 14 is inflated or not. When balloon 14 is fully inflated, as shown in FIG. 19A, ultrasound transducer 26 may be forward facing (or forward and perpendicularly facing as shown in FIG. 19B). However, when balloon 14 is deflated, ultrasound transducer 26 may be folded flat and positioned on side of distal tip 13 of sheath 12 (as balloon 14 is shown in FIGS. 1A-1C). Hence, when balloon 14 is deflated, ultrasound chip or transducer 26 may be side-facing. During inflation ultrasound transducer 26 orientation will change as balloon 14 inflates (moving from side-facing orientation to forward facing orientation with the ultrasound transducer 26 shown in FIG. 19A). Accordingly, operator(s) of transseptal insertion device 10 may vary the inflation of balloon 14 to achieve different orientations of ultrasound transducer 26 for different imaging views.

Ultrasound chip or transducers 26 may emit and/or receive/detect ultrasound waves that may be reflect off of surfaces and structures, e.g., within atrium, and then read by imaging system (not shown), e.g., connected to ultrasound chip or circuitry 26 via wire or cable extending through, e.g., lumen 15 in sheath 12. In this manner, ultrasound chip or transducers 26 may enable visualization of the interatrial septum and the left atrial structures.

It is also noted that ultrasound chips or transducers 26 may be deployed on distal tip 13 of sheath 12 (or elsewhere on or in sheath 12). Ultrasound chips or transducers 26 may be installed or configured to be forward facing (facing towards distal end of sheath 12). Alternatively, ultrasound chips or transducers 26 may be flipped to be rear facing (facing towards proximal end of sheath 12). Varying orientations of ultrasound chips or transducers 26 may be implemented.

Embodiments may include an additional balloon or dilator which would be able to dilate the distal end of sheath 12, or the entire sheath length, thereby significantly increasing the French size of the sheath 12. For example, balloons deployed within sheath 12 may be inflated to expand sheath 12. In such embodiments, transseptal insertion device 10 may, therefore, be used to accommodate and deliver larger devices or be able to retrieve devices once they have been extruded from sheath 12 and have embolized. Such balloons may be inflated through

In embodiments, energy, typically electrical energy, may directed through transseptal insertion device 10 may be used to increase or decrease the French size of sheath 12. In such embodiments, sheath 12 is fabricated from materials that are known to increase in malleability and or expand when certain energies are applied. In this manner, the French size of sheath 12 may be adjusted to a size deemed necessary during a given procedure. Such energy may be applied through wires or conductive material, connected to energy source external to proximal end of transseptal insertion device 10, attached to or fabricated within sheath 12 or other components of transseptal insertion device 10. Likewise, parts or portions of transseptal insertion device 10 may be selectively made more rigid or more malleable/soft with the application of energy. Therefore, with the application of differential energy to different parts of transseptal insertion device 10 at different times, transseptal insertion device 10 size may be adjusted to enable various devices that are ordinarily larger and bulkier than the catheter to traverse through the catheter. In embodiments, transseptal insertion device 10 may accommodate devices up to 36 Fr.

In an embodiment of transseptal insertion device 10, visualization of an intrathoracic region of interest using MRI techniques may be provided. Embodiments may, for example, provide a needle system comprising a hollow needle having a distal portion and a proximal portion, said distal portion having a distal-most end sharpened for penetrating a myocardial wall. The needle may include a first conductor, an insulator/dielectric applied to cover the first conductor over the proximal portion of said needle and a second conductor applied to cover the insulator/dielectric. The method may further direct the needle system into proximity to a myocardial wall, track progress of the needle system using active MRI tracking, penetrate the myocardial wall to approach the intrathoracic region of interest, and, use the needle system as an MRI antenna to receive magnetic resonance signals from the intrathoracic region of interest.

In related embodiments, MRI antenna may be installed on distal tip 13 of sheath 12, dilator 16 or on balloon 14, similar to ultrasound chips or transducers 26 described above. Wires connecting such MRI antenna or other MRI components may pass through lumen in dilator 16 or sheath 12 and connect with appropriate magnetic resonance energy source on exterior of distal end of transseptal insertion device 10.

Since many modifications, variations, and changes in detail can be made to the described preferred embodiments of the invention, it is intended that all matters in the foregoing description and shown in the accompanying drawings be interpreted as illustrative and not in a limiting sense. Consequently, the scope of the invention should be determined by the appended claims and their legal equivalents.

1. A transseptal insertion device which is suitable for facilitating precise and safe transseptal puncture of a cardiac interatrial septum, comprising:
a sheath that defines at least one lumen therein and has a distal end that is closest to the cardiac interatrial septum of a patient when the transseptal insertion device is in use and a proximal end that is external to the patent;
a balloon that is connected to the distal end of the sheath, wherein:
the balloon, when inflated and the transseptal insertion device is in use, overhangs and extends past the distal end of the sheath, preventing accidental puncturing of the cardiac interatrial septum and stabilizing the transseptal insertion device against fossa ovalis of the cardiac interatrial septum; and
the balloon includes one or more ultrasound transceivers that emit and receive ultrasound waves, convert the ultrasound waves to electrical signals, and transmit the electrical signals to an external imaging device that produces images of the cardiac interatrial septum from the received signals; and
a dilator that is positioned within the at least one lumen when the transseptal insertion device is in use, wherein the dilator has a distal end and is designed to and is capable of precisely puncturing the cardiac interatrial septum.

2. The transseptal insertion device of claim 1 wherein the one or more ultrasound transceivers are located on the balloon surface.

3. The transseptal insertion device of claim 1 wherein the one or more ultrasound transceivers are located inside the balloon.

4. The transseptal insertion device of claim 1 wherein the one or more ultrasound transceivers are oriented towards the cardiac interatrial septum when the balloon is fully inflated and the distal end of the sheath is oriented towards the cardiac interatrial septum.

5. The transseptal insertion device of claim 1 wherein the one or more ultrasound transceivers are oriented perpendicular to the sheath when the balloon is deflated.

6. The transseptal insertion device of claim 1 wherein the one or more ultrasound transceivers are configured in the shape of a cross so that one or more ultrasound transceivers are oriented towards the cardiac interatrial septum when the balloon is fully inflated and the distal end of the sheath is oriented towards the cardiac interatrial septum and perpendicular to the sheath.

7. The transseptal insertion device of claim 1 wherein the one or more ultrasound transceivers are configured in the shape of a disc.

8. The transseptal insertion device of claim 1 wherein the one or more ultrasound transceivers are connected to an external imaging device through a wire that runs via lumen in sheath.

9. The transseptal insertion device of claim 1 wherein the one or more ultrasound transceivers are connected to an external imaging device via WiFi or other wireless connection.

10. A transseptal insertion device which is suitable for facilitating precise and safe transseptal puncture of a cardiac interatrial septum, comprising:
a sheath that defines at least one lumen therein and has a distal end that is closest to the cardiac interatrial septum of a patient when the transseptal insertion device is in use and a proximal end that is external to the patent, wherein the sheath includes one or more ultrasound transceivers that emit and receive ultrasound waves, convert the ultrasound waves to electrical signals, and transmit the electrical signals to an external imaging device that produces images of the cardiac interatrial septum from the received signals;
a balloon that is connected to the distal end of the sheath, wherein the balloon, when inflated and the transseptal insertion device is in use, overhangs and extends past the distal end of the sheath, preventing accidental puncturing of the cardiac interatrial septum and stabilizing the transseptal insertion device against fossa ovalis of the cardiac interatrial septum; and
a dilator that is positioned within the at least one lumen when the transseptal insertion device is in use, wherein the dilator has a distal end and is designed to and is capable of precisely puncturing the cardiac interatrial septum.

11. The transseptal insertion device of claim 11 wherein the one or more ultrasound transceivers are oriented towards the distal end of the sheath.

12. The transseptal insertion device of claim 11 wherein the one or more ultrasound transceivers are oriented towards the proximal end of the sheath.

13. A transseptal insertion device which is suitable for facilitating precise and safe transseptal puncture of a cardiac interatrial septum, comprising:
a sheath that defines at least one lumen therein and has a distal end that is closest to the cardiac interatrial septum of a patient when the transseptal insertion device is in use and a proximal end that is external to the patent;
a balloon that is connected to the distal end of the sheath, wherein:
the balloon, when inflated and the transseptal insertion device is in use, overhangs and extends past the distal end of the sheath, preventing accidental puncturing of the cardiac interatrial septum and stabilizing the transseptal insertion device against fossa ovalis of the cardiac interatrial septum; and
the balloon is constructed of compliant and non-compliant material so that different portions of the balloon will expand differentially when the balloon is inflated;
a dilator that is positioned within the at least one lumen when the transseptal insertion device is in use, wherein the dilator has a distal end and is designed to and is capable of precisely puncturing the cardiac interatrial septum.

14. The transseptal insertion device of claim 13 further comprising one or more ultrasound transceivers that emit and receive ultrasound waves, convert the ultrasound waves to electrical signals, and transmit the electrical signals to an external imaging device that produces images of the cardiac interatrial septum from the received signals.

15. The transseptal insertion device of claim 14 wherein the one or more ultrasound transceivers are located on the balloon.

16. The transseptal insertion device of claim 14 wherein the one or more ultrasound transceivers are located on the dilator.

17. A transseptal insertion device which is suitable for facilitating precise and safe transseptal puncture of a cardiac interatrial septum, comprising:
a sheath that defines at least one lumen therein and has a distal end that is closest to the cardiac interatrial septum of a patient when the transseptal insertion device is in use and a proximal end that is external to the patent;
a balloon that is connected to the distal end of the sheath, wherein the balloon, when inflated and the transseptal insertion device is in use, overhangs and extends past the distal end of the sheath, preventing accidental puncturing of the cardiac interatrial septum and stabilizing the transseptal insertion device against fossa ovalis of the cardiac interatrial septum;
a dilator that is positioned within the at least one lumen when the transseptal insertion device is in use, wherein the dilator has a distal end and a lumen defined therein; and
a needle with a blunt end that is positioned within the lumen of the dilator and is designed to and is capable of precisely puncturing the cardiac interatrial septum with application of energy transmitted through the needle, wherein the needle includes malleable and rigid portions.

18. The transseptal insertion device of claim 17 in which the rigid portions of the needle include a distal end and proximal end of the needle.

19. The transseptal insertion device of claim 17 in which the energy applied by the needle is radio-frequency (RF) energy.
